(19) United States
(12) Patent Application Publication
(10) Pub. No.: US 2025/0213696 A1
(43) Pub. Date: Jul. 3, 2025

(54) HIGHLY LUMINESCENT BIODEGRADABLE UPCONVERSION NANOPROBE HAVING LONG DECOMPOSITION TIME

(71) Applicant: Research & Business Foundation SUNGKYUNKWAN UNIVERSITY, Suwon-si (KR)

(72) Inventors: Murad ABUALREJAL, Suwon-si (KR); Dong Hwan KIM, Suwon-si (KR)

(73) Assignee: Research & Business Foundation SUNGKYUNKWAN UNIVERSITY, Suwon-si (KR)

(21) Appl. No.: 18/774,558

(22) Filed: Jul. 16, 2024

(30) Foreign Application Priority Data
Jan. 3, 2024 (KR) 10-2024-0000932

(51) Int. Cl. A61K 41/00; A61K 9/51

(52) U.S. Cl. CPC A61K 41/008; A61K 9/5115; A61K 9/5192

(57) ABSTRACT

An example provides a biodegradable upconversion nanoparticle with a core-double shell structure including: a core layer; an inorganic host matrix outer shell layer; and a transition layer positioned between the core layer and the outer shell layer and functioning as an energy transfer network, wherein the core layer, the outer shell layer, and the transition layer are biodegradable. By means of such a structure, a long decomposition time and high luminescent efficiency are ensured, and thus the biodegradable upconversion nanoparticle may remain in an organism body for a long time to allow the effect to persist.

### CROSS-REFERENCE TO RELATED APPLICATION(S)

This application claims priority to Korean Patent Application No. 10-2024-0000932, filed on Jan. 3, 2024, the entire disclosure of which is incorporated herein for all aspects.

### BACKGROUND

The disclosure relates to a biodegradable upconversion nanoparticle with a core-double shell structure and a method for producing the same and, more specifically, to a biodegradable upconversion nanoparticle with a core-double shell structure having luminescence for biomedical applications and a method for producing the same.

Upconversion nanoparticles (UCNPs) doped with lanthanide elements are very promising for applications in chemotherapy, drug delivery, photothermal therapy, photodynamic therapy, biosensing, and bioassays. The unique photoluminescence properties (PL) thereof lead to the excellent visible light absorption, emission bandwidth, resistance to photobleaching, low noise, stable light scattering, and zero autofluorescence background.

β-NaYF₄:Yb³⁺/Er³⁺ is one of the most efficient phosphorus-based materials due to the excellent upconversion efficiency, high chemical stability, low lattice phonon energy, and purity phase. Recently, UCNPs have shown active and interesting research directions for various biomedical applications. UCNPs exhibited unique advantages such as high resolution, minimal background interference, negligible damage to living organisms, and deep tissue excitation and detection of fluorescent signals in vivo.

For biomedical applications, UCNPs designed with nanocomponents that are degradable and have small sizes of less than 5.5 nm required for kidney clearance are highly desirable. It is essential for the body that UCNPs be removed harmlessly from the body in a reasonable time after performing the diagnostic or therapeutic function. However, all existing UCNPs are chemically stable and physiologically inert crystal lattices.

In a previous study, biodegradable UCNCs K₃ZrF₇:Yb/Er were reported, but have disadvantages that the biodegradation time was only 8 hours and the upconversion luminescent (UCL) efficiency was very low. Therefore, it is necessary to design biodegradable UCNPs with the longest biodegradation time and high UCL efficiency.

### SUMMARY

The disclosure is to provide a biodegradable upconversion nanoparticle (UCNP) with a long biodegradation time and high UCL efficiency for biomedical applications and a method for producing the same.

The aspect of the disclosure is not limited to that mentioned above, and other aspects not mentioned will be clearly understood by those skilled in the art from the description below.

According to an example, provided is a biodegradable upconversion nanoparticle with a core-double shell structure, including: a core layer; an inorganic host matrix outer shell layer; and a transition layer positioned between the core layer and the outer shell layer and functioning as an energy transfer network, wherein the core layer, the outer shell layer, and the transition layer include nanoparticles doped with lanthanide ions, and are biodegradable, and the size of the core layer is larger than the sizes of the outer shell layer and the transition layer.

The core of the upconversion nanoparticles (UCNPs) consists of a degradable Na₃ZrF₇ matrix doped with Yb, Er/Tm, and Ca. This core emits upconversion luminescence (UCL) due to the activator ions Er/Tm, which are partially incorporated into the soft host matrix.

The middle layer, or transition layer, is composed of NaYF₄ doped with Yb and Ca. The presence of a moderate concentration of Yb in this layer facilitates efficient energy migration between the outer shell and the degradable core, helping to prevent undesired quenching effects from Nd/Yb in the outer shell.

The outer shell layer features a high concentration of Nd/Yb sensitizers within a NaY/NdF₄ matrix, also incorporating Ca. This composition is designed to absorb a high amount of near-infrared (NIR) radiation, thereby enhancing the UCL intensity.

According to another example, provided is a method for producing a biodegradable upconversion nanoparticle with a core-double shell structure, the method including: producing a first mixture by dissolving a calcium salt, a zirconium salt, and a lanthanide ion precursor in an organic solvent containing a fatty acid; dispersing the first mixture in an organic solvent containing cyclohexane; producing a second mixture by mixing a calcium salt, a yttrium salt, and a lanthanide ion precursor; adding the dispersed first mixture to the second mixture; and removing the solvent.

The lanthanide ion precursor may include two or more different lanthanide ion precursors.

The lanthanide ion precursor may include one represented by Chemical Formula 1:

Ln(CH₃COO) [Chemical Formula 1]
(In Chemical Formula 1, Ln is La, Ce, Pr, Nd, Pm, Sm, Eu, Gd, Tb, Dy, Ho, Er, Tm, Yb, or Lu.)

According to an example, an upconversion nanoparticle with a new structure of a biodegradable core layer, a transition layer, and an active outer shell layer may be provided to be applied to biomedicine.

By means of such a structure, a long decomposition time and high luminescent efficiency are ensured, and thus the biodegradable upconversion nanoparticle may remain in an organism body for a long time to allow the effect to persist.

By including a sensitizer in the outer shell layer, the luminescent intensity is enhanced, and the transition layer acts as an efficient energy transfer network between the core layer and the outer shell layer to prevent an extinction effect.

The effects of the disclosure are not limited to the effects described above, and should be understood to include all effects that are inferable from the configuration of the disclosure described in the detailed description or claims of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 schematically shows an upconversion nanoparticle including a core, core-shell, and core double-shell structure;

FIG. 2A shows a high-resolution electron microscopy (HRTEM) image for UCNP core, FIG. 2B shows HRTEM image for UCNP core-shell, FIG. 2C shows HRTEM image for UCNP core-double shell, FIG. 2D shows the brightness of a blue biodegradable core-double shell UCNP, FIG. 2E shows evaluation on the decomposition ability of a biodegradable UCNP core-double shell, FIG. 2F shows HRTEM images of a blue biodegradable UCNP core-double shell 0 h before degradation and 5, 10, 15, and 20 days after degradation;

FIG. 3A shows a high-resolution electron microscopy (HRTEM) image for UCNP core, FIG. 3B shows HRTEM image for UCNP core-shell, FIG. 3C shows HRTEM image for UCNP core-double shell, FIG. 3D shows the brightness of green biodegradable core-double shell UCNPs, FIG. 3E shows a gradual decrease in a vivid green UCL over 10 days;

FIG. 4A shows a high-resolution electron microscopy (HRTEM) image for UCNP core, FIG. 4B shows HRTEM image for UCNP core-shell, FIG. 4C shows HRTEM image for UCNP core-double shell, FIG. 4D shows the brightness of a yellow environmentally degradable core-double shell UCNPs, FIG. 4E shows a gradual decrease in vivid yellow color in a UCL over 20 days, FIG. 4F shows HRTEM images of a yellow biodegradable UCNP core-double shell 0 h before degradation and 5, 10, 15, and 20 days after degradation;

FIG. 5 shows an HAADF-STEM image and element map core analysis of a blue biodegradable core-double shell upconversion nanoparticle (UCNP);

FIG. 6 shows a result of energy dispersive X-ray spectroscopy (EDS) of a blue biodegradable core-double shell upconversion nanoparticle (UCNP) composed of Zr, Yb, Y, Na, Tm, Ca, and F elements;

FIG. 7 shows an ultrahigh-altitude rotation electron microscopy (HAADF-STEM) image and element map nuclear analysis of a green biodegradable core-double shell upconversion nanoparticle (UCNP); and

FIG. 8 shows a result of energy dispersive X-ray spectroscopy (EDS) analysis of a green biodegradable core-double shell upconversion nanoparticle (UCNP).

### DETAILED DESCRIPTION

Hereinafter, the disclosure will be described with reference to the accompanying drawings. However, the disclosure may be implemented in various different forms and, therefore, is not limited to the examples described herein. In order to clearly explain the disclosure in the drawings, portions unrelated to the description are omitted, and similar portions are given similar reference numerals throughout the specification.

Throughout the specification, when a portion is said to be “connected (linked, contacted, combined)” with another portion, this includes not only a case of being “directly connected” but also a case of being “indirectly connected” with another member in between. In addition, when a portion is said to “include” a certain component, this does not mean that other components are excluded, but that other components may be added, unless specifically stated to the contrary.

The terms used herein are merely used to describe specific examples and are not intended to limit the disclosure. Singular expressions include plural expressions unless the context clearly dictates otherwise. In this specification, it should be understood terms such as “include” or “have” are to designate the presence of features, numbers, steps, operations, components, parts, or combinations thereof described in the specification, but are not to exclude in advance the possibility of the presence or addition of one or more other features, numbers, steps, operations, components, parts, or combinations thereof.

Hereinafter, examples of the disclosure will be described in detail with reference to the accompanying drawings.

Terms used herein are defined as follows.

“Upconversion nanoparticles”, “upconverting nanoparticles”, “UCNP”, “UCNPs”, and “upward conversion nanoparticles” all have the same meaning.

“UCL”, “upconversion luminescent”, “upconversion luminescence”, “UCL intensity”, “upward conversion luminescence”, and “upward conversion fluorescence” all have the same meaning.

A biodegradable upconversion nanoparticle with a core-double shell structure according to an example is described.

According to the disclosure, the biggest problem faced by core-decomposable upconversion nanoparticles (UCNPs), that is, the lack of consistency in the shape of core-decomposable UCNPs, may be effectively resolved by conversion thereof into a consistent shape and well-distributed form.

Specifically, by coating a decomposable core via a double shell structure, the structural properties of UCNPs were improved to a more consistent and well-distributed shape.

This is possible by uniformly mixing and dissolving the different elements of a core layer, a transition layer, and an outer shell layer.

A biodegradable upconversion nanoparticle with a core-double shell structure according to an example may include: a core layer; an inorganic host matrix outer shell layer; and a transition layer positioned between the core layer and the outer shell layer and functioning as an energy transfer network, wherein the core layer, the outer shell layer, and the transition layer include nanoparticles doped with lanthanide ions, and are biodegradable, and the size of the core layer is larger than the sizes of the outer shell layer and the transition layer.

At this time, one including the transition layer and the outer shell layer may be referred to as a double shell structure.

The effect of the structural properties of a UCNP is maximized when the size of the core layer is larger than the sizes of the outer shell layer and the transition layer. In this case, the mixing of precursor materials in the core layer, transition layer, and outer shell layer is enhanced, resulting in a consistent structure, excellent optical properties, and unique chemistry, and this improves the efficacy of degradable UCNPs.

Upconversion, which is a phenomenon in which photons with lower energy are continuously absorbed and photons with higher energy are emitted, may be applied to a variety of application fields, such as improving the efficiency of solar cells by converting infrared light, which is abundant in sunlight, into visible light, or converting highly bio-permeable infrared light into visible light inside brain tissue to provide effective photostimulation. However, a conventional upconversion nanoparticle had limitations that are low upconversion efficiency and spectral purity, and had the disadvantage of having a short biodegradation time of less than about 12 hours.

On the other hand, the upconversion nanoparticle according to the disclosure has an increased biodegradation period through a multilayer structure of the core layer, transition layer, and outer shell layer, and the upconversion luminescent intensity is improved. Accordingly, the upconversion nanoparticle according to the disclosure may be applied to various biomedical fields such as bio imaging to be used more practically.

The core of the upconversion nanoparticles (UCNPs) is made up of a degradable Na₃ZrF₇ matrix infused with Yb, Er/Tm, and Ca. This core generates upconversion luminescence (UCL) due to the incorporation of activator ions Er/Tm within the soft host matrix.

The intermediate layer, also known as the transition layer, is formulated from NaYF₄ doped with Yb and Ca. The strategic placement of a moderate Yb concentration in this layer promotes effective energy transfer from the outer shell to the degradable core and minimizes the unwanted quenching effects associated with Nd/Yb in the outer shell.

The outer shell layer is composed of a NaY/NdF₄ matrix that includes a high concentration of Nd/Yb sensitizers, along with Ca. This configuration is tailored to maximize the absorption of near-infrared (NIR) radiation, which in turn enhances the intensity of the UCL.

For a method for producing an upconversion nanoparticle according to a second aspect, a detailed description of portions overlapping with a first aspect has been omitted. However, although the description is omitted, the description in the first aspect may be equally applied to the second aspect.

Hereinafter, a method for producing a biodegradable upconversion nanoparticle with a core-double shell structure according to another example will be described.

A method for producing a biodegradable upconversion nanoparticle with a core-double shell structure according to an example may include: producing a first mixture by dissolving a calcium salt, a zirconium salt, and a lanthanide ion precursor in an organic solvent containing a fatty acid; dispersing the first mixture in an organic solvent containing cyclohexane; producing a second mixture by mixing a calcium salt, a yttrium salt, and a lanthanide ion precursor; adding the dispersed first mixture to the second mixture; and removing the solvent.

First, a first mixture is produced by dissolving a calcium salt, a zirconium salt, and a lanthanide ion precursor in an organic solvent containing a fatty acid.

The lanthanide ion precursor may include two or more different lanthanide ion precursors.

The lanthanide ion precursor may include one represented by Chemical Formula 1.

Ln(CH₃COO) [Chemical Formula 1]
(In Chemical Formula 1, Ln is La, Ce, Pr, Nd, Pm, Sm, Eu, Gd, Tb, Dy, Ho, Er, Tm, Yb, or Lu.)

According to an implementation, the zirconium salt may be, but not limited to, one selected from the group consisting of zirconium (IV) acetylacetonate, zirconium acrylate, zirconium (IV) bis(diethyl citrato) dipropoxide, zirconium bromonorbornenelactone carboxylate triacrylate, zirconium (IV) butoxide, zirconium (IV) tert-butoxide, zirconium (IV) carbonate basic, zirconium carboxyethyl acrylate (in n-propanol), zirconium (IV) chloride tetrahydrofuran complex, zirconium (IV) ethoxide, zirconium (IV) isopropoxide isopropanol complex, zirconium (IV) propoxide solution (in N-propyl alcohol), zirconium (IV) 2,2,6,6-tetramethyl-3,5-heptanedionate, zirconium (IV) trifluoroacetylacetonate, and combinations thereof.

According to an implementation, the organic solvent may be, but not limited to, one selected from the group consisting of 1-octadecene, 1-nonadecene, cis-2-methyl-7-octadecene, 1-heptadecene, 1-hexadecene, 1-pentadecene, 1-tetradecene, 1-tridecene, 1-undecene, 1-dodecene, 1-decene, and combinations thereof.

According to an implementation, the fatty acid may be, but not limited to, one selected from the group consisting of an oleic acid, an elaidic acid, a gondoic acid, an erucic acid, a nervonic acid, an eicosadienoic acid, a mead acid, and combinations thereof.

The first mixture is heated, and then a methanol solution containing NH4F and NaOH is added and reacted. Thereafter, methanol is evaporated. Next, the first mixture is dispersed in an organic solvent containing cyclohexane.

Next, the second mixture is produced by mixing the calcium salt, yttrium salt, and lanthanide ion precursor, and then the dispersed first mixture is added to the second mixture.

The second mixture is added to the first mixture, and then cyclohexane is evaporated. The solvent is then removed.

Hereinafter, a production example and an experimental example of the disclosure will be described in detail.

Na₃ZrF₇:Yb,Er/Tm,Ca@NaYF₄:Yb,Ca@NaNd/YbF₄:Ca <Production example>

As for a UCNPs core, zirconium (IV) acetylacetonate (0.78 mmol L⁻¹), Yb(CH₃COO)₃(0.18 mmol L⁻¹), Ca(CH₃COOH)₂(0.1 mmol L⁻¹), and Er(CH₃COO)₃(0.02 mmol L⁻¹) or Tm(CH₃COO) 3 was mixed well in a 250 mL flask and dried by heating. Thereafter, an oleic acid (17 mL) and 1-octadecene (34 mL) were added to the flask. The mixture was heated to 150° C. under Ar atmosphere to obtain a homogeneous solution. After cooling to 50° C., a methanol solution (10 mL) containing NH4F (4 mmol) and NaOH (2.5 mmol) was added to a solution and reacted for 30 minutes. Methanol was evaporated, and degassing was performed for 10 minutes. Thereafter, the temperature of the mixture was increased to 300° C. at a rate of 10° C. min-1 and incubation was performed at 300° C. for 60 min. After cooling to room temperature under ambient conditions, the as-prepared UCNPs (named NaZrF4:Yb, Er, Ca UCNPs) were collected by centrifugation (10,000 rpm for 10 min) and washed with ethanol (10 mL, three times). Finally, additional experiments were performed by redispersing NaZrF₄:Yb, Er, Ca UCNPs in cyclohexane (10 mL).

For the synthesis of core-shell UCNPs, 100 mL of Y(CH₃COO)₃(0.7 mmol L⁻¹), Yb(CH₃COO)₃(0.2-0.4 mmol L⁻¹, 0.3), and Ca(CH₃COOH)₂(0.1 mol L⁻¹) was added to a 100 mL flask and dried by heating. The mixture was heated to 150° C. under Ar atmosphere to obtain a homogeneous solution. After cooling to 80° C., Na₃ZrF₇:Yb, Er, Ca UCNP (1 mmol) in cyclohexane (10 mL) was added to the mixture. After removing cyclohexane by evaporation, a methanol solution (10 mL) containing NH4F (4 mmol) and NaOH (2.5 mmol) was added and stirred at 50° C. for 30 minutes. Methanol was evaporated, and degassing was performed for 10 minutes. Thereafter, the temperature of the mixture was increased to 300° C. at a rate of 10° C. min-1 and incubation was performed at 300° C. for 60 min. The following procedure for an NasZrF₇:Yb, Er, Ca@NaYF₄:Yb, Ca UCNP was the same as that for a core nanoparticle.

For the synthesis of NasZrF₇:Yb, Er, Ca@NaYF₄:Yb, Ca@NaNd/YF₄:Yb, Ca UCNPs, an NaNdF₄:Yb, Ca outer growth procedure was the same as the synthesis of NaYF₄:Yb,Ca UCNPs, except that Nd(CH₃COO)₃(0.8 mol L⁻¹) was used.

### EXPERIMENTAL EXAMPLE

FIG. 1 schematically shows an upconversion nanoparticle including a core, core-shell, and core double-shell structure.

Referring to FIG. 1, two different models which are a “non-uniformly resolved core” model A and a “mixed” model B and C are shown, and in A, there are chemically and structurally well-defined heterogeneous forms with large dimensions. On the other hand, in B and C, shell growth results in an even distribution of chemically and structurally uniform shapes, and characterization is made by interfaces scattered at various scales, and thus chemically and structurally heterogeneous forms are shown.

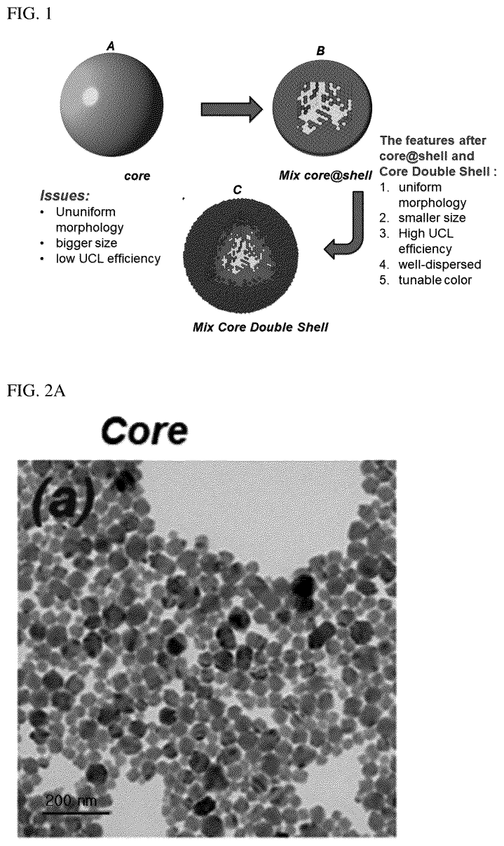

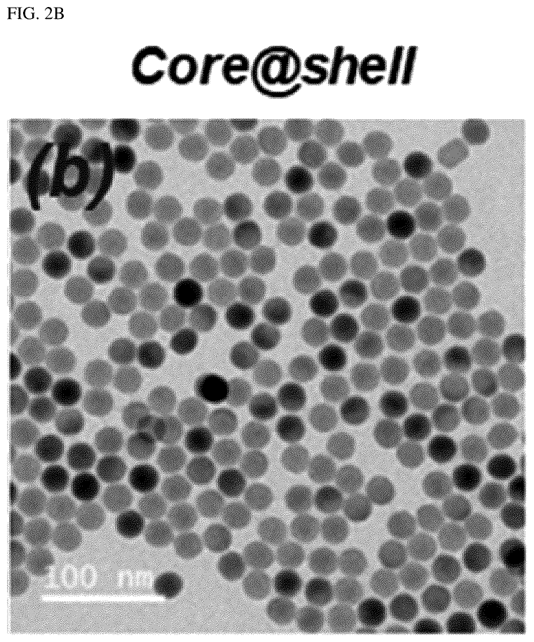

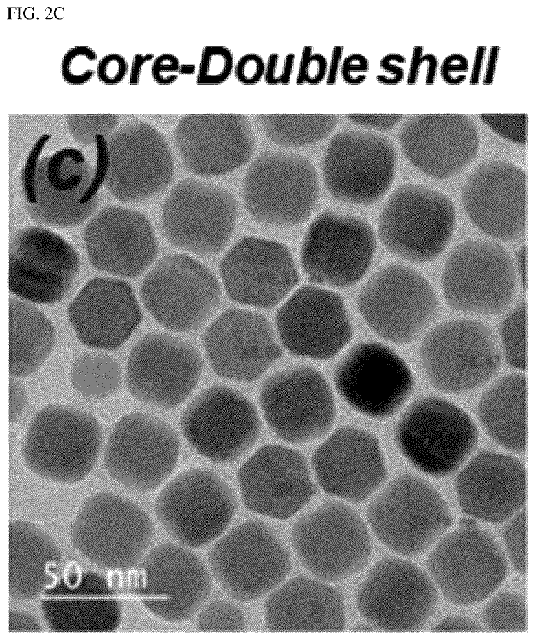

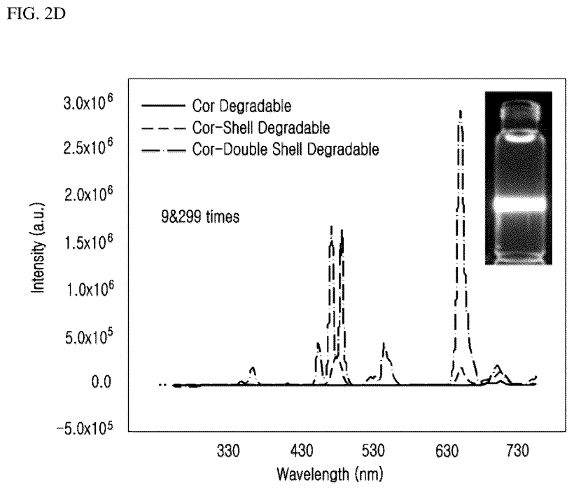

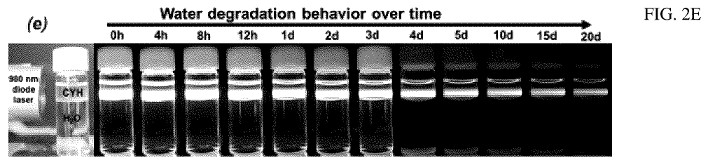

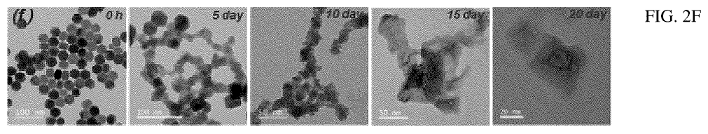

FIG. 2 shows a high-resolution electron microscopy (HRTEM) image for three types of biodegradable upconversion nanoparticles.

Referring to FIG. 2, a biodegradable UCNP core (a), a biodegradable UCNP core-shell (b), and a biodegradable UCNP core-double shell (c) show high purity of a biodegradable UCNP and were produced in high yield without side effects. The biodegradable UCNP core-double shell is uniformly monodispersed and has an average diameter of 28±1 nm. (d) shows the brightness of a blue biodegradable core-double shell UCNP. The UCL intensity (blue line) of a blue biodegradable UCNP core-double shell is 299 times higher than that of a biodegradable UCNP core (black line) and 9 times higher than that of a biodegradable UCNP core-shell (red line) at 478 nm. (e), which is evaluation on the decomposition ability of a biodegradable UCNP core-double shell, shows that 1 ml of a biodegradable UCNP core-double shell dispersed in cyclohexane was added to 3 ml of ion-exchanged water over time. (e) shows a gradual loss of a bright blue UCL within 10 days and this loss continues. Accordingly, it was confirmed that it is possible to degrade a biodegradable UCNP core-double shell for a long period of several days compared to 8 hours for K₃ZrF₇:Yb, Er biodegradable UCNPs, the state-of-the-art material.

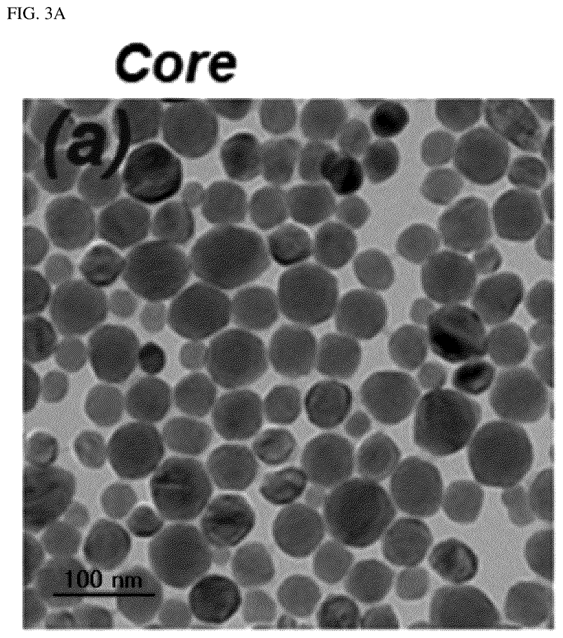

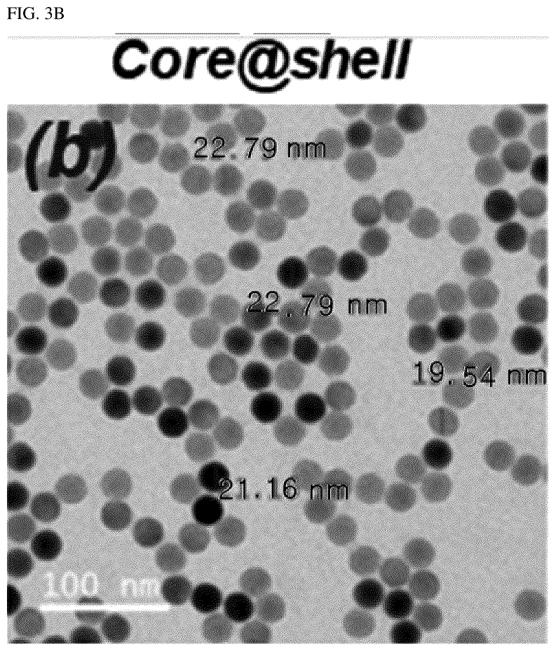

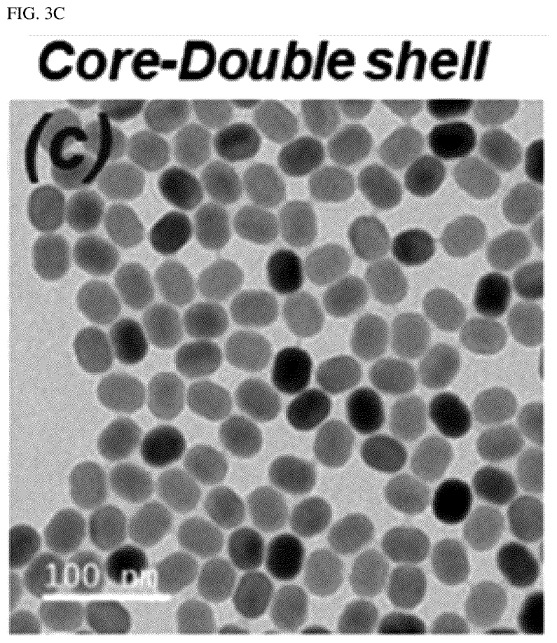

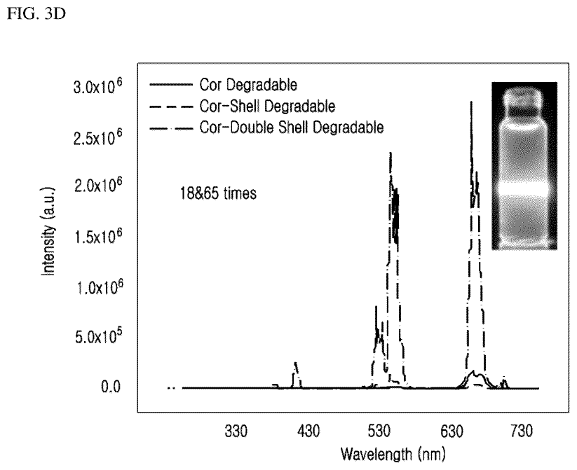

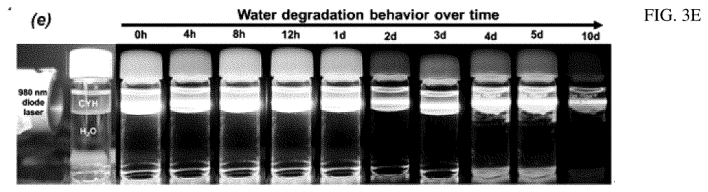

FIG. 3 shows an HRTEM image for three different types of biodegradable UCNPs.

Referring to FIG. 3, the high yield of a biodegradable UCNP without unwanted by-products indicates the high purity of a biodegradable UCNP core (a), a biodegradable UCNP core-shell (b), and a biodegradable UCNP core-double shell (c). The biodegradable UCNP core-double shell shows uniform and single dispersion, with an average diameter of 33±2 nm. (d) shows the brightness of green biodegradable core-double shell upconversion nanoparticles (UCNPs), and the UCL intensity of a green biodegradable UCNP core-double shell (blue line) is 65 nm higher than that of a biodegradable UCNP core (black line) and 18 times higher than that of a biodegradable UCNP core-shell (red line) at 541 nm. Evaluation of the degradability of a biodegradable UCNP core-double shell was performed by adding 1 ml of a dispersed UCNP to cyclohexane and adding the same to 3 ml of deionized water. Decomposition was monitored over time. (e) shows a gradual decrease in a vivid green UCL over 10 days, and this decrease persists. This enables to confirm that a green biodegradable UCNP with a core-double shell structure of a biodegradable UCNP has a longer decomposition time of several days than K₃ZrF₇:Yb, Er biodegradable UCNCs, the state-of-the-art material.

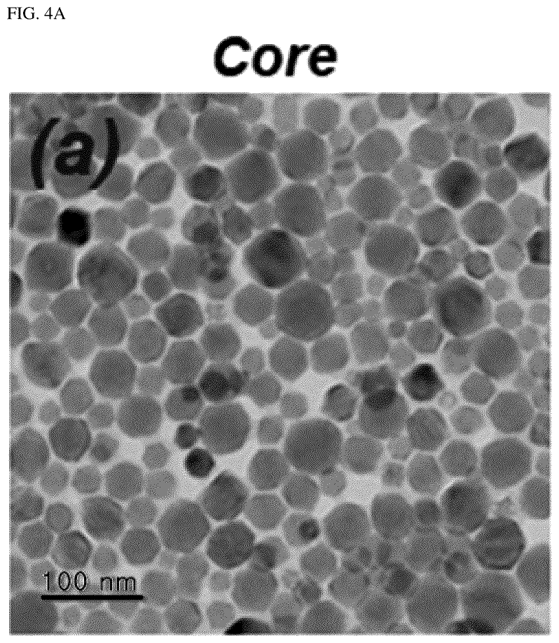

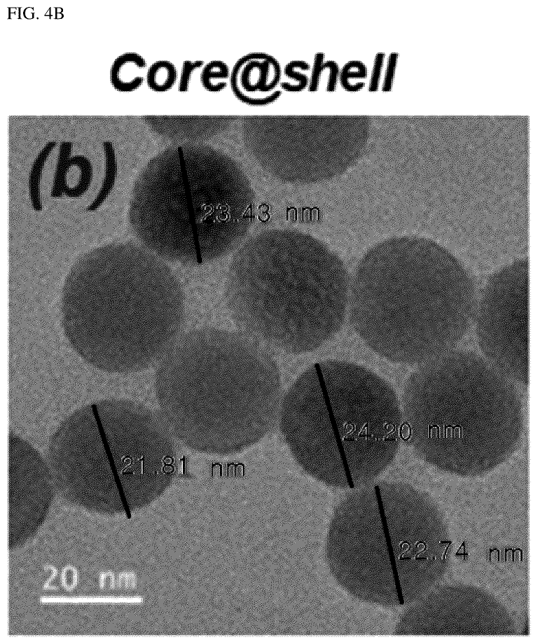

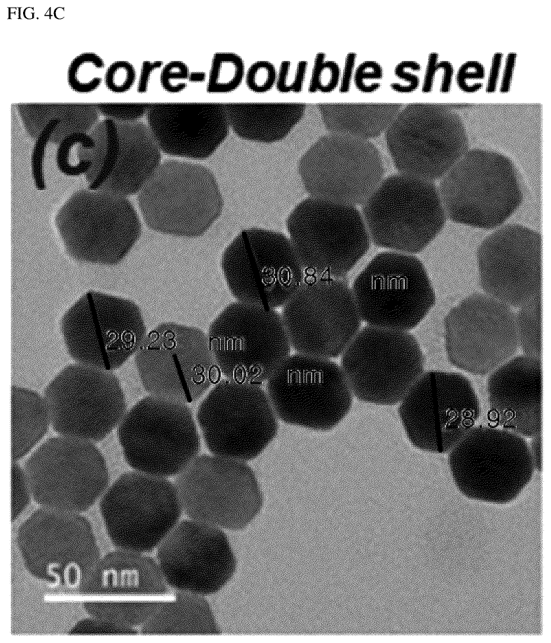

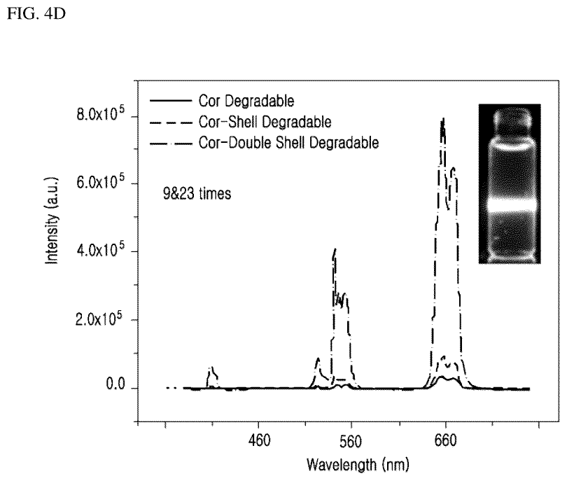

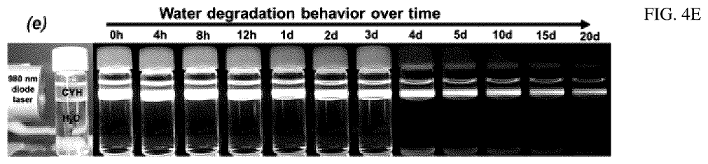

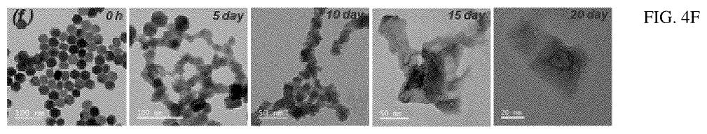

FIG. 4 shows a high-resolution transmission electron microscopy (HRTEM) image for three types of biodegradable upconversion nanoparticles.

Referring to FIG. 4, it is possible to confirm the excellent purity of a biodegradable UCNP core (a), a biodegradable UCNP core-shell (b), and a biodegradable UCNP core-double shell (c). The biodegradable UCNP core-double shell has biodegradable properties and shows uniform and single dispersion with an average diameter of 30±0.5 nm. (d) shows the brightness of a yellow environmentally degradable core-double shell upconversion nanoparticles (UCNPs). The yellow biodegradable UCNP core-double shell has a UCL intensity that is 23 times higher than that of a biodegradable UCNP core and 9 times higher than that of a biodegradable UCNP core-shell at 654 nm. In order to investigate the degradability of a biodegradable UCNP core-double shell, 1 ml of a dispersed UCNP was added to cyclohexane and then added to 3 ml of deionized water, and the degradation process that proceeded was observed and recorded over time. (e) shows a gradual decrease in vivid yellow color in a UCL over 20 days, and this decrease continued. (f) shows HRTEM images of a yellow biodegradable UCNP core-double shell 0 h before degradation and 5, 10, 15, and 20 days after degradation. It is possible to confirm that a yellow biodegradable upconversion nanoparticle (UCNP) has a core-double shell structure, resulting in a significantly long degradation period in contrast to a material K₃ZrF₇:Yb, Er biodegradable upconversion nanocrystal (UCNC) decomposed in just 8 hours.

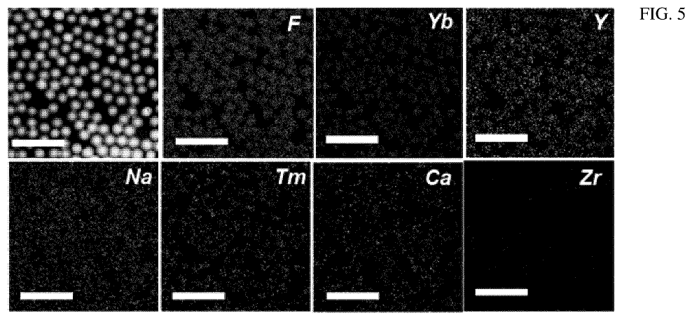

FIG. 5 shows an HAADF-STEM image and element map core analysis of a blue biodegradable core-double shell upconversion nanoparticle (UCNP).

Referring to FIG. 5, the form of a biodegradable UCNP core-double shell with Zr, Yb, Y, Na, Tm, Ca, and F uniformly distributed in diameter is shown, and the indicated scale line is 250 nm.

FIG. 6 and Table 1 show a result of energy dispersive X-ray spectroscopy (EDS) of a blue biodegradable core-double shell upconversion nanoparticle (UCNP) composed of Zr, Yb, Y, Na, Tm, Ca, and F elements.

TABLE 1
Map Sum Spectrum

| Element | Line Type | Wt % | Wt % Sigma | Atomic % |
|---|---|---|---|---|
| F | K series | 36.21 | 0.46 | 70.11 |
| Na | K series | 7.43 | 0.21 | 11.88 |
| Ca | K series | 3.93 | 0.13 | 3.61 |
| Y | K series | 15.77 | 0.51 | 6.53 |
| Zr | K series | 0.33 | 0.24 | 0.13 |
| Tm | L series | 3.18 | 0.40 | 0.69 |
| Yb | L series | 33.15 | 0.54 | 7.05 |
| Total: | | 100.00 | | 100.00 |

Referring to FIG. 6 and Table 1, it is possible to confirm the properties of a blue biodegradable core-double shell upconversion nanoparticle (UCNP) composed of Zr, Yb, Y, Na, Er, Ca, and F elements.

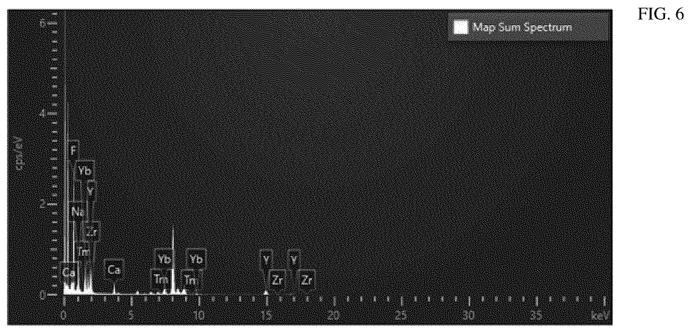

FIG. 7 shows an ultrahigh-altitude rotation electron microscopy (HAADF-STEM) image and element map nuclear analysis of a green biodegradable core-double shell upconversion nanoparticle (UCNP).

Referring to FIG. 7, it is possible to confirm zirconium (Zr), ytterbium (Yb), yttrium (Y), sodium (Na), erbium (Er), calcium (Ca), and fluoride (F), all of which have uniformly distributed diameters, and thus it is possible to see the form of a core-double shell upconversion nanoparticle (UCNP) thereof. The scale line provided is 100 nm.

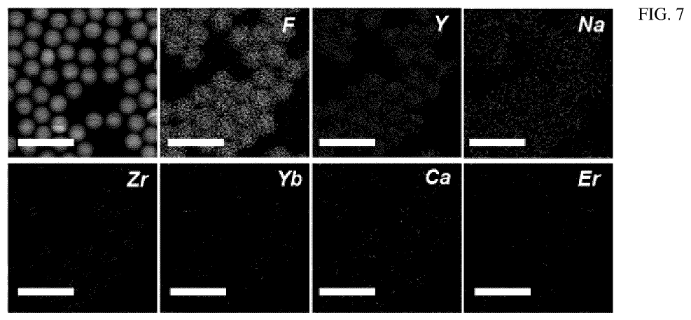

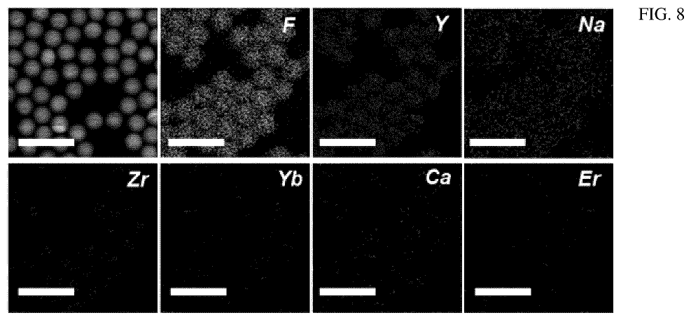

FIG. 8 and Table 2 show a result of energy dispersive X-ray spectroscopy (EDS) analysis of a green biodegradable core-double shell upconversion nanoparticle (UCNP).

TABLE 2
Map Sum Spectrum

| Element | Line Type | Wt % | Wt % Sigma | Atomic % |
|---|---|---|---|---|
| F | K series | 50.34 | 0.81 | 75.95 |
| Na | K series | 8.19 | 0.28 | 10.21 |
| Ca | K series | 2.74 | 0.14 | 1.96 |
| Y | K series | 34.25 | 0.77 | 11.04 |
| Zr | K series | 0.57 | 0.37 | 0.18 |
| Er | L series | 1.07 | 0.95 | 0.18 |
| Yb | L series | 2.84 | 0.44 | 0.47 |
| Total: | | 100.00 | | 100.00 |

Referring to FIG. 8 and Table 2, it is possible to confirm the properties of a green biodegradable core-double shell upconversion nanoparticle (UCNP) composed of Zr, Yb, Y, Na, Er, Ca, and F elements.

The UCNP according to the disclosure is biodegradable, which means that natural decomposition thereof within the body is possible over time. This is important for biomedical applications because this ensures that a UCNP is eliminated from the body after performing the diagnostic or therapeutic function.

In addition, a UCNP has a long-term decomposition time (days). This is important for biomedical applications because this allows a UCNP to remain in the body long enough to perform the essential function.

Furthermore, the UCNP according to the disclosure has adjustable luminescence color, which means that it is possible to control the color of light emitted therefrom. This is important for biomedical applications because this allows a UCNP to be used for a variety of usages such as imaging, diagnosis, and therapy.

The description of the disclosure described above is for illustrative purposes, and those skilled in the art will understand that the disclosure is easily modifiable into other specific forms without changing the technical idea or essential features of the disclosure. Therefore, the examples described above should be understood in all respects as illustrative and not restrictive. For example, each component described as single may be implemented in a distributed manner, and similarly, components described as distributed may also be implemented in a combined form.

The scope of the disclosure is indicated by the claims described below, and all changes or modified forms derived from the meaning and scope of the claims and their equivalent concepts should be construed as being included in the scope of the disclosure.

What is claimed is:

1. A biodegradable upconversion nanoparticle with a core-double shell structure, comprising:
a core layer;
an inorganic host matrix outer shell layer; and
a transition layer positioned between the core layer and the outer shell layer and functioning as an energy transfer network,
wherein the core layer, the outer shell layer, and the transition layer comprise nanoparticles doped with lanthanide ions, and are biodegradable, and
the size of the core layer is larger than the sizes of the outer shell layer and the transition layer.

2. The biodegradable upconversion nanoparticle with a core-double shell structure of claim 1, wherein the core layer comprises Na₃ZrF₇:Yb, Er/Tm, Ca nanoparticles.

3. The biodegradable upconversion nanoparticle with a core-double shell structure of claim 1, wherein the core layer exhibits UCL intensity.

4. The biodegradable upconversion nanoparticle with a core-double shell structure of claim 1, wherein the outer shell layer comprises a sensitizer.

5. The biodegradable upconversion nanoparticle with a core-double shell structure of claim 1, wherein the outer shell layer absorbs a near-infrared ray (NIR) to enhance UCL intensity.

6. The biodegradable upconversion nanoparticle with a core-double shell structure of claim 1, wherein the outer shell layer comprises NaY/NdF₄:Yb, Ca nanoparticles.

7. The biodegradable upconversion nanoparticle with a core-double shell structure of claim 1, wherein the transition layer comprises NaYF₄:Yb, Ca nanoparticles.

8. A method for producing a biodegradable upconversion nanoparticle with a core-double shell structure, the method comprising:
producing a first mixture by dissolving a calcium salt, a zirconium salt, and a lanthanide ion precursor in an organic solvent containing a fatty acid;
dispersing the first mixture in an organic solvent containing cyclohexane;
producing a second mixture by mixing a calcium salt, a yttrium salt, and a lanthanide ion precursor;
adding the dispersed first mixture to the second mixture; and
removing the solvent.

9. The method of claim 8, wherein the lanthanide ion precursor comprises two or more different lanthanide ion precursors.

10. The method of claim 8, wherein the lanthanide ion precursor comprises one represented by Chemical Formula 1:
Ln(CH₃COO) [Chemical Formula 1]
(In Chemical Formula 1, Ln is La, Ce, Pr, Nd, Pm, Sm, Eu, Gd, Tb, Dy, Ho, Er, Tm, Yb, or Lu).
